(19) United States
(12) Patent Application Publication
(10) Pub. No.: US 2004/0053848 A1
(43) Pub. Date: Mar. 18, 2004

(54) Antibodies specific for methylated lysines in histones

(76) Inventors: C. David Allis, Charlottesville, VA (US); Brian D. Strahl, Chapel Hill, NC (US)

Correspondence Address: UNIVERSITY OF VIRGINIA PATENT FOUNDATION, 1224 WEST MAIN STREET, SUITE 1-110, CHARLOTTESVILLE, VA, 22903, US

(21) Appl. No.: 10/344,878

(22) PCT Filed: Aug. 23, 2001
(86) PCT No.: PCT/US01/26283

(51) Int. Cl. A61K 39/395; A61K 38/10; C07K 7/08; C07K 16/18

(52) U.S. Cl. 514/14; 530/326; 530/388.1; 424/130.1

(57) ABSTRACT

The present invention related to the generation of methyllysine-specific histone antibodies. In particular, the H3 lysine 4 methylation specific antibody (Methyl(K4)H3) binds to histone H3 methylated at lysine 4. Methylation of lysine 4 (K4) on histone H3 has been associated with transcriptionally active regions of chromatin. A second antibody, H3 lysine 9 methylation specific antibody (Methyl(K9)H3) specifically binds to histone H3 methylated at lysine 9. Methylation of lysine 9 (K9) on histone H3 has been associated with gene silencing. These antibodies are useful in identifying regions of heterochromatin and euchromatin and serving as diagnostic and screening tools.

This application claims priority under 35 U.S.C. §119(e) to U.S. Provisional Patent Application No. 60/302,747, filed on Jul. 3, 2001 and U.S. Provisional Patent Application No. 60/227,767, filed on Aug. 25, 2000, the disclosures of which are incorporated herein by reference in their entirety.

### US GOVERNMENT RIGHTS

This invention was made with United States Government support under Grant Nos. RO1 GM40922 and RO1 GM53512, awarded by the National Institutes of Health. The United States Government has certain rights in the invention.

### FIELD OF THE INVENTION

The present invention is directed to antibodies that bind to histone epitopes created by postranslational modification of the histone protein, compositions comprising such antibodies, and the use of such compositions as diagnostic and screening tools.

### BACKGROUND OF THE INVENTION

In eukaryotes, DNA is complexed with histone proteins to form nucleosomes, the repeating subunits of chromatin. This packaging of DNA imposes a severe restriction to proteins seeking access to DNA for DNA-templated processes such as transcription or replication. It is becoming increasingly clear that post-translational modifications of histone amino-termini play an important role in determining the chromatin structure of the eukaryotic cell genome as well as regulating the expression of cellular genes.

Posttranslational modifications of histone amino-termini have long been thought to play a central role in the control of chromatin structure and function. A large number of covalent modifications of histones have been documented, including acetylation, phosphorylation, methylation, ubiquitination, and ADP ribosylation, that take place on the amino terminus “tail” domains of histones. Such diversity in the types of modifications and the remarkable specificity for residues undergoing these modifications suggest a complex hierarchy of order and combinatorial function that remains unclear. Of the covalent modifications known to take place on histone amino-termini, acetylation is perhaps the best studied and appreciated. Recent studies have identified previously characterized coactivators and corepressors that acetylate or deacetylate, respectively, specific lysine residues in histones in response to their recruitment to target promoters in chromatin (See Berger (1999) Curr. Opin. Genet. Dev. 11, 336-341). These studies provide compelling evidence that chromatin remodeling plays a fundamental role in the regulation of transcription from nucleosomal templates.

Chromosomes in higher eukaryotes have historically been considered to consist of regions of euchromatin and heterochromatin, which are distinguished by the degree of condensation and level of transcriptional activity of the underlying DNA sequences. Certain regions of constitutive heterochromatin are found at or near specialized structures such as centromeres, and are comprised mostly of genetically inert repetitive sequences. In contrast, other regions that have the same primary DNA sequences can exhibit characteristics of either type of chromatin, suggesting that epigenetic factors, such as packaging of DNA by histones and chromatin associated proteins, dictate the heterochromatin status at these loci.

Through the use of antibodies that specifically recognize histones bearing specific post-translational modifications applicants have been elucidating a “histone code.” In particular, evidence is emerging that histone proteins, and their associated covalent modifications, contribute to a mechanism that can alter chromatin structure, thereby leading to inherited differences in transcriptional “on-off” states or to the stable propagation of chromosomes by defining a specialized higher-order structure.

Histone methylation is one of the least-understood posttranslational modifications affecting histones. Early work suggests that H3 and H4 are the primary histones modified by methylation, and sequencing studies, using bulk histones, have shown that several lysines (e.g., 9 and 27 of H3 and 20 of H4) are often preferred sites of methylation, although species-specific differences appear to exist. Interestingly, each modified lysine has the capacity to be mono-, di-, or trimethylated, adding yet another level of variation to this posttranslational “mark”. The present invention is directed to antibodies that are specific for histone H3 and H4 that are methylated at specific lysines. More particularly, one aspect of the present invention is directed to histone H3 lysine 4 and 9. These two lysine residues are found to be methylated in vivo and the methylated forms are associated with euchromatin and heterochromatin, respectively.

Definitions

In describing and claiming the invention, the following terminology will be used in accordance with the definitions set forth below.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

The term “peptide” encompasses a sequence of 3 or more amino acids wherein the amino acids are naturally occurring or synthetic (non-naturally occurring) amino acids. Peptide mimetics include peptides having one or more of the following modifications:

1. peptides wherein one or more of the peptidyl —C(O)NR— linkages (bonds) have been replaced by a non-peptidyl linkage such as a —CH2-carbamate linkage (—CH2OC(O)NR—), a phosphonate linkage, a —CH2-sulfonamide (—CH 2—S(O)2NR—) linkage, a urea (—NHC(O)NH—) linkage, a —CH2-secondary amine linkage, or with an alkylated peptidyl linkage (—C(O)NR—) wherein R is C1-C4 alkyl;

2. peptides wherein the N-terminus is derivatized to a —NRR1 group, to a —NRC(O)R group, to a —NRC(O)OR group, to a —NRS(O)2R group, to a —NHC(O)NHR group where R and R1 are hydrogen or C1-C4 alkyl with the proviso that R and R1 are not both hydrogen;

3. peptides wherein the C terminus is derivatized to —C(O)R2 where R 2 is selected from the group consisting of C1-C4 alkoxy, and —NR3R4 where R3 and R4 are independently selected from the group consisting of hydrogen and C1-C4 alkyl.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Vat or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

As used herein, the term “conservative amino acid substitution” is defined herein as exchanges within one of the following five groups:

I. Small aliphatic, nonpolar or slightly polar residues:

Ala, Ser, Thr, Pro, Gly;

II. Polar, negatively charged residues and their amides:

Asp, Asn, Glu, Gln;

III. Polar, positively charged residues:

His, Arg, Lys;

IV. Large, aliphatic, nonpolar residues:

Met Leu, Ile, Val, Cys

V. Large, aromatic residues:

Phe, Tyr, Trp

As used herein, the term “purified” and like terms relate to the isolation of a molecule or compound in a form that is substantially free of contaminants normally associated with the molecule or compound in a native or natural environment.

“Operably linked” refers to a juxtaposition wherein the components are configured so as to perform their usual function. For example, control sequences or promoters operably linked to a coding sequence are capable of effecting the expression of the coding sequence.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

“Therapeutic agent,” “pharmaceutical agent” or “drug” refers to any therapeutic or prophylactic agent which may be used in the treatment (including the prevention, diagnosis, alleviation, or cure) of a malady, affliction, disease or injury in a patient.

As used herein, the term “treating” includes alleviating the symptoms associated with a specific disorder or condition and/or preventing or eliminating said symptoms. For example, treating cancer includes preventing or slowing the growth and/or division of cancer cells as well as killing cancer cells.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “antigenic fragment of H3 lysine 4” or “antigenic fragment of H3 lysine 4” encompasses both natural peptide fragments of the amino terminus of Histone 3 (including the peptide fragments of SEQ ID NO: 1, SEQ ID NO: 2 and SEQ ID NO: 3) and synthetic equivalents of those fragments.

As used herein, the term “antibody” refers to a polyclonal or monoclonal antibody or a binding fragment thereof such as Fab, F(ab′)₂ and Fv fragments.

As used herein, the term “biologically active fragments” of the Methyl(K4)H3 or Methyl(K9)H3 antibodies encompasses natural or synthetic portions of the respective full-length antibody that are capable of specific binding to the peptide of SEQ ID NO: 4 or SEQ ID NO: 5, respectively.

As used herein, the term “parenteral” includes administration subcutaneously, intravenously or intramuscularly.

As used herein the letter K in bold face type (K), when used in the context of an amino acid sequence, will represent a lysine amino acid that has been methylated.

### SUMMARY OF THE INVENTION

The present invention is directed to antibodies that bind to specific modifications of the amino terminus of histone H3 and H4 peptides. More particularly, the present invention is directed to the generation of methyllysine-specific histone antibodies. These antibodies recognize lysine residues in histones H3 and H4 that are specifically methylated and may be linked to human biology and disease. Compositions comprising these antibodies are used as diagnostic and screening tools.

### BRIEF DESCRIPTION OF THE DRAWINGS

FIGS. 1A and 1B represent immunofluorescence patterns of human metaphase chromosomes from the normal female cell line (HH) stained with either the Methyl(K9)H3 antibody (FIG. 1A) or the Methyl(K4)H3 antibody (FIG. 1B). Localization of the Methyl(K4)H3 and Methyl(K9)H3 antibodies was detected using Cy3-conjugated secondary antibody (red). Each of the immunofluorescence patterns obtained with the two antibodies revealed one chromosome that is preferentially stained compared to the other chromosomes (indicated by large arrow). As shown in FIG. 1B only small regions of the inactive X chromosome are enriched for Lys4 methyl H3 staining.

FIG. 2. CHIP analysis of somatic cell hybrid cell lines. Chromatin from CHO somatic hybrid cells containing the inactive (X_{inactive}) or active (X_{active}) human X chromosome was immunoprecipitated using the following antibodies:

lane 1 = no antibody (control lane);
lane 2 = Methyl(K9)H3;
lane 3 = Methyl(K4)H3;
lane 4 = Lys9/14-acetylated H3;
lane 5 = no DNA (control);
lane 6 = genomic DNA

The presence of Xist and PGK1 promoter DNA sequences in the immunoprecipitated DNA was assayed by PCR. The PCR products were separated on 15% polyacrylamide gels, imaged by digital camera, and images were electronically inverted to facilitate visualization of the ethidium bromide-stained bands. As shown, from cells containing the inactive X chromosome, the Methyl(K4)H3 and Lys9/14 acetyl H3 antibodies preferentially immunoprecipitated the active Xist gene whereas the Methyl(K9)H3 antibody preferentially immunoprecipitated the inactive PGK1 gene. From cells containing the active X chromosome, the exact reverse immunoprecipitation pattern was observed.

FIG. 3 Immunoblot analysis of H3 methylation between simple vs. complex organisms. Histones were isolated from various sources, and five ug of total core histones from each species, along with 1 ug of recombinant Xenopus H3 were resolved on a 15% SDS-PAGE, transferred to a PVDF membrane support and probed with either the Methyl(K4)H3 or Methyl(K9)H3 antibody. Lanes 1-5 represent histones isolated from recombinant Xenopus H3, budding yeast, Tetrahymena, chicken and the human cell line 293T, respectively. Identical samples were analyzed in parallel and examined by Coomassie staining to monitor histone loading.

### DETAILED DESCRIPTION OF THE INVENTION

Histone methylation is a poorly understood post-translational modification affecting histones. This modification occurs on selected lysine residues in the amino-terminus of histones. It is now becoming apparent that methylating histone enzymes are involved in both gene activation and repression. The present invention is directed to the generation of methyllysine-specific histone antibodies. These antibodies recognize lysine residues in the histones H3 and H4 that are specifically methylated and may be linked to human biology and disease.

The present invention is directed to post-translational modifications that occur on the flexible N-terminal tails of the core histone proteins H3 and H4. More particularly, the invention is directed to methylated lysine residues. Applicants have discovered that methylation of the lysine residues within the first 15 amino acids of the amino terminus of H3 (SEQ ID NO: 7) and H4 (SEQ ID NO: 8) play an important role in the regulation of transcription. For example, methylation of lysine 4 (K4) on histone H3 has been associated with transcriptionally active regions of chromatin, whereas methylation of lysine 9 (K9) on histone H3 has been associated with gene silencing. Therefore, in accordance with one aspect of the present invention methylation of lysine 4 (K4) and lysine 9 (K9) on histone H3 serve as a markers of euchromatin and heterochromatin, respectively, and antibodies recognizing these modified proteins have use as important diagnostic tools.

One aspect of the present invention is directed to antigens used to produce antibodies specific to the amino terminus of H3 and H4. In one embodiment, the antigen is a purified antigenic fragment of the amino terminus of H3 or H4 methlated at a lysine and selected from the group consisting of ARTKQTARKSTGG (SEQ ID NO: 10), ARTKQTARKSTGG (SEQ ID NO: 1), ARTKQTARKSTGV (SEQ ID NO: 12), ARTKQTARKSTGV (SEQ ID NO: 13), SGRGKGGKGLGKG (SEQ ID NO: 14) and SGRGKGGKGLGKG (SEQ ID NO: 15) or a synthetic equivalent thereof, wherein the bold K represents a methylated lysine residue. In one embodiment the antigen comprises an H3 amino terminal fragment of 20 amino acids or less and comprises the sequence ARTKQTAR (SEQ ID NO: 1), QTARKSTGV (SEQ ID NO: 2) or QTARKSTGG (SEQ ID NO: 3), and derivatives of these amino acid sequences wherein the amino acid sequence contains one or more conservative amino acid substitutions. In one preferred embodiment the antigen is ARTKQTAR (SEQ ID NO: 1), QTARKSTGV (SEQ ID NO: 2) or QTARKSTGG (SEQ ID NO: 3), or a derivative thereof containing additional non-native amino acids added to either end of the peptide sequence.

In an alternative embodiment, the purified antigen comprises a polypeptide linked to a suitable carrier, such as bovine serum albumin or Keyhole limpet hemocyanin. In one preferred embodiment the antigen consists of an H3 peptide fragment peptide comprising a sequence selected from the group ARTKQTAR (SEQ ID NO: 1), QTARKSTGV (SEQ ID NO: 2) or QTARKSTGG (SEQ ID NO: 3, and derivatives of this amino acid sequence wherein the amino acid sequence contains one or more conservative amino acid substitutions, and a carrier protein linked to the peptide. For example, the antigen may comprise a peptide having the sequence ARTKQTARGC (SEQ ID NO: 4), QTARKSTGVCG (SEQ ID NO: 5), QTARKSTGGCG (SEQ ID NO: 6), AARKSAPVCG (SEQ ID NO: 16), SGGVKKPHKCG (SEQ ID NO: 17) or RHKILRDCG (SEQ ID NO: 18) wherein the bold K represents the methylated lysine residue and the underlined GC refers to artificial amino acids added to the natural histone sequence. Furthermore, the antigen can optionally be linked to a carrier protein.

In addition to the antigens described above, the present invention is also directed to antibodies that specifically bind to peptide fragments of the H3 or H4 protein that have been methylated at a lysine residue. Preferably the antibody will recognize one or more methylated lysine residues present in the first 20 amino acid residues of the amino terminus of the H3 and H4 histones. More particularly, the present invention is directed to an antibody that specifically binds to the peptide ARTKQTARGC (SEQ ID NO: 4), QTARKSTGGCG (SEQ ID NO: 6), QTARKSTGVCG (SEQ ID NO: 5), AARKSAPVCG (SEQ ID NO: 16), SGGVKKPHKCG (SEQ ID NO: 17) or RHRKLRDCG (SEQ ID NO: 18), wherein the bold K represents the methylated lysine residue and the underlined GC refers to artificial amino acids added to the natural histone sequence to aid in the production of the antibody. In one embodiment the antibody is specific for a peptide comprising the amino acid selected from the group consisting of ARTKQTARGC (SEQ ID NO: 4), QTARKSTGVCG (SEQ ID NO: 5), QTARKSTGGCG (SEQ ID NO: 6); and amino acid sequences that differ from SEQ ID NO: 4, SEQ ID NO: 5, SEQ ID or SEQ ID NO: 6 by one or more conservative amino acid substitutions. In one preferred embodiment the antibody specifically binds to the peptide ARTKQTARGC (SEQ ID NO: 4) or QTARKSTGVCG (SEQ ID NO: 5).

Antibodies that specifically bind an H3 peptide that is methylated at lysine 4 (i.e. the peptide of SEQ ID NO: 4) will be referred to as Methyl(K4)H3 and antibodies that specifically bind an H3 peptide that is methylated at lysine 9 (i.e. the peptide of SEQ ID NO: 5 or SEQ ID NO: 6) will be referred to as Methyl(K9)H3. These two antibodies do not cross react and will not bind to the non-methylated peptide sequences. The present invention also encompasses antibodies that bind to the non-methylated histone peptides. In one embodiment, the antibodies of the present invention are monoclonal antibodies.

The antibodies or antibody fragments of the present invention can be combined with a carrier or diluent to form a composition. In one embodiment, the carrier is a pharmaceutically acceptable carrier. Such carriers and diluents include sterile liquids such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose, and related sugar solution, and glycols such as, propylene glycol or polyethylene glycol, are preferred liquid carriers, particularly for injectable solutions. The compositions comprising the Methyl(K4)H3 or Methyl(K9)H3 antibody, or bioactive fragments thereof, and a carrier or diluent can be used in conjunction with the method to detect heterochromatin verses euchromatin.

One method used to generate the antibodies of the present invention involves administration of an antigen, comprising the sequence ARTKQTAR (SEQ ID NO: 1), QTARKSTGV (SEQ ID NO: 2) or QTARKSTGG (SEQ ID NO: 3), to a laboratory animal, typically a rabbit, to trigger production of antibodies specific for the antigen. The dose and regiment of antigen administration to trigger antibody production as well as the methods for purification of the antibody are well known to those skilled in the art. Typically, such antibodies can be raised by administering the antigen of interest subcutaneously to New Zealand white rabbits which have first been bled to obtain pre-immune serum. The antigens can be injected at a total volume of 100 ul per site at six different sites. Each injected material will contain synthetic surfactant adjuvant pluronic polyols, or pulverized acrylamide gel containing the protein or polypeptide after SDS-polyacrylamide gel electrophoresis.

The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbital 150 mg/Kg IV. This and other procedures for raising polyclonal antibodies are disclosed in E. Harlow, et. al., editors, Antibodies: A Laboratory Manual (1988), which is hereby incorporated by reference. The specificity of antibodies may be determined by enzyme-linked immunosorbent assay or immunoblotting, or similar methods known to those skilled in the art.

The present invention also encompasses monoclonal antibodies that specifically bind to the respective antigen ARTKQTAR (SEQ ID NO: 1), QTARKSTGV (SEQ ID NO: 2) or QTARKSTGG (SEQ ID NO: 3) and their non-methylated counterpart peptides. Monoclonal antibody production may be effected using techniques well-known to those skilled in the art. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. One embodiment of the invention is directed to a hybridoma cell line which produces monoclonal antibodies which bind the methyl-lysine peptides of the present invention. A description of the theoretical basis and practical methodology of fusing such cells is set forth in Kohler and Milstein, Nature, 256:495 (1975), which is hereby incorporated by reference.

In addition to whole antibodies, fragments of antibodies can retain binding specificity for a particular antigen. Antibody fragments can be generated by several methods, including, but not limited to proteolysis or synthesis using recombinant DNA technology. An example of such an embodiment is selective proteolysis of an antibody by papain to generate Fab fragments, or by pepsin to generate a F(ab′)₂ fragment. These antibody fragments can be made by conventional procedures, as described in J. Goding, Monoclonal Antibodies: Principles and Practice, pp. 98-118 N.Y. Academic Press 1983), which is hereby incorporated by reference. Other fragments of the present antibodies which retain the specific binding of the whole antibody can be generated by other means known to those skilled in the art.

In one embodiment the antibodies are labeled. It is not intended that the present invention be limited to any particular detection system or label. The antibody may be labeled with a fluorophore, a radioisotope, or a non-isotopic labeling reagent such as biotin or digoxigenin; antibodies containing biotin may be detected using “detection reagents” such as avidin conjugated to any desirable label such as a fluorochrome. In one embodiment the histone specific antibodies of the present invention are detected through the use of a secondary antibody, wherein the secondary antibody is labeled and is specific for the primary (histone specific) antibody. Alternatively, the histone specific antibody may be directly labeled with a radioisotope or fluorochrome such as FITC or rhodamine; in such cases secondary detection reagents may not be required for the detection of the labeled probe.

In accordance with one embodiment of the present invention a method of detecting the presence of methylated lysine residues in the H3 and H4 histones is provided. The method comprises the steps of contacting histone proteins with a labeled antibody, wherein the antibody specifically binds only to H3 that is methylated at lysine 4 or H3 methylated at lysine 9.

In accordance with one embodiment the antibodies of the present invention are labeled for use in diagnostic imaging. Examples of labels useful for diagnostic imaging in accordance with the present invention are radiolabels such as ¹³¹I, ¹¹¹In, ¹²³I, ^{99m}Tc, ³²P, ¹²⁵I, ³H, ¹⁴C, and ¹⁸⁸Rh fluorescent labels such as fluorescein and rhodamine, nuclear magnetic resonance active labels, electron dense or radiopaque materials, positron emitting isotopes detectable by a positron emission tomography (“PET” ) scanner, chemilluminescers such as luciferin, and enzymatic markers such as peroxidase or phosphatase. Short-range radiation emitters, such as isotopes detectable by short-range detector probes, such as a transrectal probe, can also be employed. These isotopes and transrectal detector probes, when used in combination, are especially useful in detecting prostatic fossa recurrences and pelvic nodal disease. The antibodies of the present invention can be labeled with such reagents using techniques known in the art For example, see Wensel and Meares, Radioimmunoimaging and Radioimmunotherapy, Elsevier, New York (1983), which is hereby incorporated by reference, for techniques relating to the radiolabeling of antibodies. See also, D. Colcher et al., “Use of Monoclonal Antibodies as Radiopharmaceuticals for the Localization of Human Carcinoma Xenografts in Athymic Mice,” Meth. EnzvmQL, 121: 802-816 (1986), which is hereby incorporated by reference. In accordance with one preferred embodiment the antibody is labeled with a fluorophore or chromophore using standard moieties known in the art.

Applicants have discovered that methylation of histone H3 at lysines 4 and 9 (K4 and K9), correlates, respectively, with activation and inactivation of expression of genes in proximity to the modified histones. Histone H3 when methylated at K9 is a preferred binding site for the heterochromatin protein HP1, which in turn can recruit the enzyme Suv39h responsible for K9 methylation, creating a mechanism by which the inactivation signal can be propagated. Methylation of K9 also precludes acetylation at that site, further contributing to repression. In contrast to the reaction at K9, methylation at histone H3 K4 is correlated with transcriptional activity. Therefore in accordance with one embodiment of the present invention, antibodies specific for the K4 methylated histone H3 can be used to detect transcriptionally active regions of chromatin and antibodies specific for K9 methylated histone H3 transcriptionally can be used to detect inactive regions of chromatin. In fact, in situ staining of chromosomes reveals that the staining patterns generated by the Methyl(K4)H3 and Methyl(K9)H3 antibodies produce mirror images of one another.

Because the Methyl(K4)H3 and Methyl(K9)H3 antibodies have the potential for use in humans as diagnostic and therapeutic agents, one embodiment of the present invention is directed to humanized versions of the Methyl(K4)H³ and Methyl(K9)H3 antibodies. Humanized versions of the antibodies are needed for therapeutic applications because antibodies from non-human species may be recognized as foreign substances by the human immune system and neutralized such that they are less useful. Humanized antibodies are immunoglobulin molecules comprising a human and non-human portion. More specifically, the antigen combining region (variable region) of a humanized antibody is derived from a non-human source (e.g. murine) and the constant region of the humanized antibody is derived from a human source. The humanized antibody should have the antigen binding specificity of the non-human antibody molecule and the effector function conferred by the human antibody molecule. Typically, creation of a humanized antibody involves the use of recombinant DNA techniques.

The antibodies of the present invention can also be linked to an insoluble support to provide a means of isolating euchromatin or heterochromatin from cells. The support may be in particulate or solid form and could include, but is not limited to: a plate, a test tube, beads, a ball, a filter or a membrane. Methods for fixing antibodies to insoluble supports are known to those skilled in the art. In one embodiment an antibody of the current invention is fixed to an insoluble support that is suitable for use in affinity chromatography.

The Methyl(K4)H3 and Methyl(K9)H3 antibodies are large-scale or domain-sensitive chromatin marks that are somehow set up by boundary elements. In particular, chromatin that is associated with histones that include H3 methylated at Lsy4 represent an “on” domain, or at least a domain that is competent for transcriptional activity. Alternatively, chromatin that is associated with histones that include H3 methylated at Lsy9 represent an “off” domain, that is not competent for transcriptional activity. This pattern is conserved across a diverse range of species. Zoo blots with these antibodies suggest that most of the H3 histones from ‘simple’ organisms (budding yeast) and Tetrahymena, contain a methylated Lys4 (ON), whereas in striking contrast, most of the H3 histones present in ‘complex’ organisms have a methylated Lys9 (OFF) (see FIG. 3). This agrees well with the finding that most of the genomic DNA in yeast and in Tetrahymena is expressed (ON) while most of the DNA in humans, mice, etc. is OFF. Thus, it may well be that the ‘default’ or ground state in more complex eukaryotes is OFF. Knowing how to identify ON chromatin through use of the Lys4 methyl mark may prove invaluable in developing strategies for better targeting of transgene to more ‘friendly’ chromatin.

Chromatin immunoprecipitation data supports the above ‘ON/OFF’ marking system model. In particular, S. pombe chromatin IP data, published (Science Nakayama et al., 2001) and the work on the inactive X chromosome in humans (see Example 2) supports this model. Furthermore there are ‘hot spots’ of Lys4 H3 methylation on the ‘inactive’ X chromosome and in some cases tumor supressor genes have been mapped into this chromosomal region. Loss of heterozygosity at this gene correlates well with a significant number of advanced cases of ovarian cancers. Therefore the present antibodies can also be used as diagnostics for detecting cancer and for determining therapy strategies. In accordance with one embodiment a method is provided for detecting chromatin alterations that are associated with a disease state. The term “disease state” is intended to encompass any condition that is associated with an impairment of the normal state of a living animal or plant including congenital defects, pathological conditions such as cancer, and responses to environmental factors and infectious agents (bacterial, viral, etc.). The method comprises the steps of isolating chromatin from both normal and diseased tissue, contacting the two pools of chromatin with either the Methyl(K4)H3 or Methyl(K9)H3 antibody and comparing the staining pattern of the chromatin isolated from normal tissue to that of the diseased tissue. Furthermore, using chromatin immunoprecipitation, unique tumor suppressor genes could be isolation by differential screening using the antibodies of the present invention.

In accordance with one embodiment of the present invention the Methyl(K4)H3 and Methyl(K9)H3 antibodies are used to identify heterochromatin and euchromatin regions and thus detect transcriptionally active and inactive regions of chromatin. More particularly, the antibodies can be used to detect changes in chromatin structure that are associated with a given disease state. Therefore the antibodies can be used as a diagnostic to detect alterations of chromatin structure that are associated alterations in expression patterns (i.e. differences in heterochromatin vs euchromatin patterns relative to predominant native patterns). Alterations in chromatin structure for a specific region of chromatin may be diagnostic of a particular disease state. For example, conversion of a normally euchromatic region of the genome to heterochromatin may represent the suppression of a tumor suppressor gene that is indicative of cancer or a pre-cancer state. Similarly the conversion of a region of heterochromatin to euchromatin may be associated with the inappropriate or overexpression of a gene that has deleterious effects on the host cell/organism.

The present invention is also directed to a method of using broad-based differential screening techniques to isolate nucleic acid regions that have altered expression patterns in diseased tissues. For example, chromatin can be isolated from diseased tissues and compared to chromatin isolated from healthy tissues to determine if there are any differences in the chromatin structure (i.e. changes in heterochromatin vs. euchromatin) that are associated with the disease state. Such differences in chromatin structure may represent suppression or overexpression of genes that play a direct or indirect role in the disease. The anti-methyl(Lys 9) H3 and anti-methyl(Lys 4) H3 antibodies can be used to detect such changes in chromatin structure and help identify genes that are associated with the disease state. The identification of such genes will assist in designing more effective therapies for treating the disease.

In one embodiment the method for detecting alterations in chromatin structure associated with a particular disease comprises chromatin immunoprecipitation assays, using modification-specific histone antobodies. This process allows for the analysis of a wide range of DNA-templated processes that are governed by the chromatin environment. More particularly, the method comprises the steps of isolating chromatin from both diseased tissue and healthy tissue, fragmenting the DNA (preferably by sonification), and immunoprecipitating chromatin using an antibody that specifically binds to the amino acid sequence of ARTKQTAR (SEQ ID NO: 1) or QTARKSTGV (SEQ ID NO: 2), wherein the bold K represents a methylated lysine residue, and comparing the chromatin (and the associated DNA sequences) immunoprecipitated from the healthy tissue relative to the diseased tissue. Comparison of the two pools of immunoprecipitated chromatin will allow for the identification of differences between diseased and healthy tissues.

In one embodiment, comparison of the two pools of immunoprecipitated chromatin comprises the steps of isolating the nucleic acid sequences associated with the two pools of immunoprecipitated chromatin and comparing the resulting two pools of nucleic acid sequences. Comparison of the two pools of nucleic acid sequences can be conducted using any of the standard molecular techniques, including PCR, gel electrophoresis, nucleic acid sequencing and nucleic acid hybridization analysis. Those nucleic acid sequences that are present in only one of the two pools of nucleic acid sequences are then recovered. These nucleic acid sequences represent expressed/suppressed genes that are associated with either the normal or diseased tissue. In one embodiment the antibodies used to immunoprecipitate the chromatin are selected from the group consisting of Methyl(K4)H3 and Methyl(K9)H3 antibody.

The use of methyl Lys4/Lys9 histone H3 antibodies in chromatin immunoprecipitation (chromatin IP) assays is one way to enrich for genomic DNA corresponding to the epigenetic ‘ON/OFF’ state of the human genome (and other genomes as well). By combining chromatin immunoprecipitated DNA with current genomic microarray technology (on chips), one has the potential to survey any portion of the human (or other) genome as to their on/off state through the ‘histone code’. For example, DNA immunoprecipitated using the Methyl(K4)H3 antibody can be immobilized on a solid surface or “chip” and thus represent all the nucleic acid sequences of a given cell that is competent for transcription. Similarly, DNA immunoprecipitated using the Methyl(K9)H3 antibody can be immobilized on a solid surface or “chip” and thus represent all the nucleic acid sequences of a given cell that is not competent for transcription. Harvesting mRNA or preparing cDNA from a target cell, labeling the target nucleic acids and then hybridizing the target DNA with the immobilized DNA will reveal abnormal expression of genes.

Knowing this information may prove invaluable in determining the on/off state of key tumor suppressor or oncogenic proteins in various human cancers. Knowing how this epigenetic marking corresponds to genomic DNA will also guide the ability to produce transgenic animals and plants where one often finds that most transgenic DNA enters a ‘bad’ (Lys9) chromatin environment and is silenced. Thus, the implications for knowing how to better ‘guide’ DNA into a ‘good’ (Lys4) chromatin environment for animal and plant transgenic work are high. In humans, this would impact on gene therapy issues as well.

In one embodiment, immunoprecipitation of chromatin will be used to map the location of active genes at a genome-wide level through the use of microarrays. For example, in one preferred embodiment the method of comparing the two pools of immunoprecipitated chromatin (i.e. the immunoprecipitated chromatin from diseased vs healthy tissues) comprises the use of a gene chip, DNA microarray, or a proteomics chip using standard techniques known to those skilled in the art. For example any of the systems described in WO 01/16860,WO 01/16860, WO 01/05935, WO 00/79326, WO 00/73504, WO 00/71746 and WO 00/53811 (the disclosures of which are expressly incorporated herein) are suitable for use in the present invention. Preferably the chip will contain an ordered array of known compounds, such as known

DNA sequences, so that interaction of the immunoprecipitated chromatin at a specific location of the chip will identify, and allow for the isolation of, DNA sequences associated with the immunoprecipitated chromatin.

The key to this technology is the use of antibodies specific to various modification as they relate to the histone code. Applying this to human and other genomes would lay the foundation of epigenomics. While the present invention has detailed the use of Lys4/Lys9 methyl H3 antibodies as respective ON/OFF antibodies, this concept applies more generally to any and all antibodies that are developed directed at the ‘histone code’. For example, Lys9 methyl vs. Ser10 phos H3 antibodies may also be a ‘methyl/phos’ switch that regulates differentiation vs. proliferation. The present invention also encompasses antibodies that are directed to other methylated regions of the amino terminus of H3 and H4 histones, including H3 lysines 27 and 36 and H4 lysine 20. The peptides that will be used to generate these antibodies are listed below:

| | | |
|---|---|---|
| H3 lysine 27: | AARKSAPVCG | (SEQ ID NO:16) |
| H3 lysine 36: | SGGVKKPHKCG | (SEQ ID NO:17) |
| H4 lysine 20: | RHRKILRDCG | (SEQ ID NO:18) |

wherein the bold K is the methylated lysine residue and underlined GC refers to artificial amino acids added to the H3 sequence to aid in the production of this antibody.

The antibodies of the present invention can be used in standard Molecular Biology techniques such as Western blot analyses, immunofluorescence, and immunoprecipitation. As noted above the presence of methylated H3 at lysine 4 correlates with transcriptionally active nuclei, and therefore, this H3 antibody may be a useful in the understanding of gene regulation. In addition it is anticipated that microinjection of the Methyl(K4)H3 antibody into cells may interfere with the activation of specific genes.

In one embodiment of the present invention a kit is provided for detecting euchromatin and heterochromatin. The kit comprises an antibody that specifically binds to a lysine methlyated modified peptide selected from the group consisting of ARTKQTARGC (SEQ ID NO: 4), QTARKSTGVCG (SEQ ID NO: 5), ARTKQTAR (SEQ ID NO: 1), QTARSTGV (SEQ ID NO: 2), ARTKQTARKSTGV (SEQ ID NO: 9), AARKSAPVCG (SEQ ID NO: 16), SGGVKKPHKCG (SEQ ID

NO: 17) and RHRKILRDCG (SEQ ID NO: 18). More particularly, the kit comprises an antibody that binds to the peptide ARTKQTARGC (SEQ ID NO: 4), QTARKSTGVCG (SEQ ID NO: 5) or QTARKSTGGCG (SEQ ID NO: 6), wherein the bold K represents a methylated lysine residue. In one embodiment the antibodies are attached to an insoluble support, wherein the support is either a monolithic solid or is in particular form. In one preferred embodiment the antibodies are monoclonal antibodies and in a further embodiment the antibodies are labeled. To this end, the antibodies of the present invention can be packaged in a variety of containers, e.g., vials, tubes, microtiter well plates, bottles, and the like. Other reagents can be included in separate containers and provided with the kit; e.g., positive control samples, negative control samples, buffers, cell culture media, etc.

In an another embodiment of the invention a kit is provided for use in an assay to determine if a sample has methylase activity. The kit comprises a peptide selected from the group consisting of ARTKQTARGC (SEQ ID NO: 4), QTARKSTGVCG (SEQ ID NO: 5), ARTKQTAR (SEQ ID NO: 1), QTARSTGV (SEQ ID NO: 2) and ARTKQTARKSTGV (SEQ ID NO: 9) and an antibody that specifically binds to a lysine methlyated modified peptide selected from the group consisting of ARTKQTARGC (SEQ ID NO: 4), QTARKSTGVCG (SEQ ID NO: 5), ARTKQTAR (SEQ ID NO: 1), QTARSTGV (SEQ ID NO: 2) and ARTKQTARKSTGV (SEQ ID NO: 9). In one embodiment the antibodies are attached to an insoluble support, wherein the support is either a monolithic solid or is in particular form. In another embodiment the kit is further provided with an antibody that specifically binds to a non-methylated peptide selected from the group consisting of ARTKQTARGC (SEQ ID NO: 4), QTARKSTGVCG (SEQ ID NO: 5), ARTKQTAR (SEQ ID NO: 1), QTARSTGV (SEQ ID NO: 2) and ARTKQTARKSTGV (SEQ ID NO: 9). Such an antibody serves as a negative control.

In one embodiment, the method for detecting the methylase activity of a sample comprises contacting a peptide selected from the group consisting of ARTKQTARGC (SEQ ID NO: 4), QTARKSTGVCG (SEQ ID NO: 5), ARTKQTAR (SEQ ID NO: 1), QTARSTGV (SEQ ID NO: 2) and ARTKQTARKSTGV (SEQ ID NO: 9) for a predetermined length of time with a sample that is suspected of having methylase activity. The amount of methylation-specific antibody (i.e. Methyl(K4)H3 or Methyl(K9)H3) that binds to the substrate is a direct correlation of the extent the substrate was methylated during the predetermined time length and thus indicates the methylase activity of the sample. This assay can also be used to screen for potential inhibitors of a methylase. For example, in one embodiment a method of screening for inhibitors of arginine methyl transfer activity comprises the steps of providing a sample, wherein the sample comprises a methylase and a substrate that is methylated by said methylase, adding a potential inhibitor of the methylase to the sample, and contacting the sample with an antibody that binds specifically to the methylated substrate, but not the non-methylated substrate. In one embodiment, the antibody is specific for the peptide ARTKQTAR (SEQ ID NO: 1) or QTARKSTGV (SEQ ID NO: 2). Quantifying the amount of antibody bound to the peptide is a direct correlation of the level activity of the methylase in the sample. In one preferred embodiment the methylase activity to be detected is SuVar3-9 (for Lys 9) or Set1 (for Lys 4).

‘Knock-out’ strains are available for all of the non-essential genes present in budding yeast . . . around 4,800. The antibodies of the present invention have such a high degree of specificity, that they only detect one or two major bands in yeast whole cell lysates, thus allowing for the development of a robotic screening method to look at all of these knock-out strains. Using Lys4 methyl H3 should lead to the entire upstream pathway of ‘regulators’ including the on and off enzymes (provided the gene product is non-essential).

Using the above approach in ‘old-fashioned’ blots, is has been found that Set1 is the enzyme responsible for the Lys4 H3 methyl mark in yeast, and is also one of the Lys4 HMTases in humans. Surprisingly, using the methyl Lys4 H3 antibodies as a readout to probe yeast whole cell lysates, Lys4 methylation was discovered to be regulated by H2B ubiquitination at a conserved Lysine on the opposite side of the nucleosome. This is the first example of a ‘trans-tail’ effect meaning that one histone modification on one tail effect another modification on another not-so-close tail. In humans and in mice, the enzymes that add ubiquitin to proteins is human Rad6 (HR6), and HR6 comes in two isoforms, HR6A and HR6B. Mice knockouts of HR6B −/− are male sterile in a pathway that is not known, but seems to lead to chromatin defects during spermatogenesis leading to sperm death. Accordingly it is anticipated that HR6B is responsible for ubiquitin addition on H2B and therefore it is possible that Lys4 H3 methyl antibodies will be a diagnostic for male infertility. Furthermore, the possibility exists that defects in Lys9 methylation could impact X-inactivation and lead to female infertility. Therefore in accordance with one embodiment of the present invention the Methyl(K4)H3 and Methyl(K9)H3 antibodies are used as a diagnostic to screen for male and female infertility defects.

Current models suggest that Lys9 methylation is catalzyed, at least in some instances by SuVar3-9. It appears that the Lys9 methyl mark is read by chromodomains, short protein modules that act as chromatin ‘velcro’ patches. Best documented is the chromodomain of the heterochromatin protein HP 1. Interestingly, the chromodomain from HP1, binds well to Lys9 methyl H3 peptides, but binds much less well to Lys4 or unmodified peptides. It is interesting also that SuVar3-9 itself, a catalytic HMTase, also has a chromodomain, a module whose site specificity for methylated histone peptides has yet to be tested.

It seems likely that the Lys4 methyl mark in H3 will be read by a distinct chromodomain from that of HP1. In one embodiment the uniquely-modified peptides of the present invention, including ARTKQTAR (SEQ ID NO: 1) or QTARKSTGV (SEQ ID NO: 2) are used as affinity reagents to look for polypeptides that bind Lys4 H3 peptides. Several attractive candidates include two histone acetyltransferases (HATs), Esa1 and CDY, both of whom have chromodomains. Interestingly CDY is a testis-specific HAT encoded on the male Y chromosomes, and somatic histones are well known to be hyperacetylated during a reaction that leads to replacement by protamines.

It could be that during spermatogenesis the following series of concerted reactions occur:

1. H2B ubiquitination happens, catalyzed by HR6B

2. Chromatin opening

3. Lys4 methylation catalyzed by human Set1 or another Lys4 HMTase

4. Binding of the HAT CDY to the Lys4 methylation mark though its chromodomain

5. Histone hyperacetylation occurs catalyzed by CDY

6. Somatic histones are displaced, followed by transition proteins, followed by protamines

Defects in any of the above steps could lead to sperm lethality and male infertility.

Note that three of the steps require chromatin-modifying enzymes:

i) HR6B a E2 ubiquitin-conjugating enzyme

ii) Set1 or a resposible Lys4 HMTase

iii) CDY a male-specific HAT

Antibodies to ubiquitinated H2B and H2A, Lys4 methyl H3 and sites of CDY-catalyzed acetylation would all be of potential diagnostic value in male infertility screens.

### EXAMPLE 1

Preparation of the Methyl(K4)H3 and the Methyl(K9)H3 Antibodies

To generate the Methyl(K4)H3 and the Methyl(K9)H3 antibody, a short polypeptide corresponding to the amino-acid sequence of histone H3 surrounding lysine 4 (SEQ ID NO: 4; ARTKQTARGC) or lysine 9 (SEQ ID NO: 5; QTARKSTGVCG) was first chemically synthesized, wherein the bold K is the methylated lysine residue and underlined GC refers to artificial amino acids added to the H3 sequence to aid in the production of this antibody. This polypeptide was then conjugated to cationized bovine serun albumin (BSA), and the conjugated-peptide was injected into rabbits. One important aspect to this procedure is the fact that the standard technique of conjugating a peptide to Keyhole Limpet Hemocyanin (KLH) proved ineffective in generating high quality methyllysine 4 (H3)-specific antisera. Thus, the use of this specialized cationized BSA is now considered a unique “method” in generating methyllysine-specific antibodies.

Rabbit serum was harvested at regular intervals post-immunization, and the Methyl(K4)H3 and Methyl(K9)H3 antibodies were shown to be present in the Serum by standard enzyme-linked immunosorbent assays. These antibodies are suitable for use in Western blot, immunofluorescence, and immunoprecipitation assays.

### EXAMPLE 2

Role of Histone H3 Lysine 9 Methylation in Epigenetic Control of Heterochromatin Assembly

The assembly of higher order chromatin structures has been linked to the covalent modifications of histone tails. In vivo evidence demonstrates that lysine 9 of histone H3 (H3 Lys 9) is preferentially methylated by the Clr4 protein at heterochromatin-associated regions in fission yeast. Both the conserved chromo- and SET domains of Clr4 are required for H3 Lys 9 methylation in vivo.

The organization of the higher order chromatin structure has been linked to the posttranslational modifications of histone tails, including acetylation, phosphorylation, and methylation. It has been suggested that distinct combinations of covalent histone modifications, also referred to as the “histone code,” provide a “mark” on the histone tails to recruit downstream chromatin-modifying proteins. This is best illustrated by recent studies indicating that the conserved bromo-domain of several transcriptional coactivators bind specifically to acetylated lysine residues on histone tails. The mechanisms responsible for the establishment and maintenance of multiple covalent modifications within the same or different histone tail are not fully understood.

Modifications of histone tails have also been linked to heterochromatin assembly. Histones H3 and H4 are largely hypoacetylated in heterochromatic chromosomal regions in organisms as diverse as yeast, flies, and mammals. In fission yeast, hypoacetylation of histones is associated with the silent mating-type region and centromeres, chromosomal domains that share many parallels with heterochromatic regions in higher eukaryotes. Centromeric regions comprising a central core of unique sequences surrounded by inner (imr) and outer (otr) repeats are assembled into silenced chromatin structures. Similarly, a large; 15-kb chromosomal domain at the mating-type (mat2/3) region, including the mat2 and mat3 loci and an interval between them, known as the K-region, is maintained in a silent epigenetic state. Among the trans-acting factors that affect silencing at these regions, Clr3 and Clr6 belong to family of histone deacetylases (HDACs). Swi6 and Clr4 proteins contain a chromodomain, an evolutionarily conserved motif initially identified in HP1 and Polycomb proteins. Recently, both Clr4 and its mammalian counter-part, SUV39H1, have been shown to have intrinsic histone H3-specific methyltransferase (HMTase) activity in vitro (S. Rea et al., Nature 406, 593 (2000)). However, it is not known whether histones are the physiological targets of these methyltransferases in vivo.

Consistent with previous findings, recombinant Clr4 (rClr4) was found to contain HMTase activity exclusively for histone H3. To identify the specific residue of H3 methylated by rClr4, synthetic peptides derived from the NH 2-terminus of H3 were used as substrates in an in vitro HMTase assay. In particular, five milligrams of HeLa or chicken core histones was incubated with 0.55 mCi of S-adenosyl-L-[methyl-3H]methionine (3H-AdoMet; 72 Ci/mmol; 1 mM Dnal) and 2 mg of recombinant Clr4 wild-type or mutant proteins in 25 ml of HMTase buffer [50 mM tris (pH 8.0), 1 mM phenylmethylsulfonyl fluoride, 0.5 mM dithiothreitol in 10% glycerol] for 1 hour at 30° C. SDS loading buffer was added to half of each sample and boiled followed by separation on a 15% SDS-polyacrylamide gel electrophoresis (PAGE) gel. The resulting histone bands were visualized by Coomassie staining and fluorography. For the peptide analysis, 5 mg of each peptide derived from the NH₂-terminus of human histone H3 containing a COOH-terminal cysteine was used. Half of the sample was spotted on Whatman P-81 filter paper and washed four times for 10 min in 50 mM NaHCO₂ (pH 9.0), followed by liquid scintillation counting. (3. D. Strahl, R. Ohba, R. G. Cook, C. D. Allis, Proc. Natl. Acad Sci. U.S.A. 96,14967 (1999)). Clr4 preferentially methylated the H3 1-20 unmodified peptide but failed to methylate the H3 19-35 unmodified peptide, indicating that the target residue of Clr4 HMTase resides in the first 20 amino acids of H3.

To determine this target residue, a synthetic H3 1-20 peptide set was developed that contained covalent modifications on different amino acids. With these peptides as substrates, only acetyl or methyl modifications on Lys 9 effectively blocked rClr4 HMTase activity, indicating that Clr4, like its mammalian homolog SUV39H1, selectively methylates Lys 9 of H3. Furthermore, similar to SUV39H1, rClr4 HMTase activity was inhibited by phosphorylation of serine 10. These results demonstrate that enzymatic features of the Su(var)3-9 protein family are evolutionarily conserved from fission yeast to humans. A recent study demonstrated that the conserved SET domain and two flanking cysteine-rich regions were required for SUV39H1 HMTase activity in vitro. To determine whether the conserved domains, the chromo, SET, and cysteine-rich regions, were also critical for Clr4 HMTase activity, mutant Clr4 proteins were tested for HMTase activity. Although mutations in the chromo-domain [Trp³¹ to Gly (W31G) and Trp⁴¹ to Gly (W41G)] had little effect on Clr4 HMTase activity, mutations in the SET domain [Gly³²⁸ to Ser (G378S)] and both cysteine-rich regions [Arg320 to His (R320H) and Gly⁴⁸⁶ to Asp (G486D)] greatly reduced Clr4 HMTase activity, indicating that these three regions are critical for Clr4 HMTase activity in vitro.

To test the hypothetical correlation between H3 Lys 9 methylation and silencing, an H3 Lys 9-methyl specific antibody was developed. In an enzyme-linked immunosorbent assay, the H3 Lys 9-methyl antibody specifically recognized the H3 1-20 Lys 9-methyl peptide in a wide range of antibody dilution. Moreover, the H3 Lys 9-methyl antibody did not detect recombinant histone H3 (rH3) alone compared with the HeLa core histone positive control but did detect rH3 selectively methylated by rClr4, further demonstrating the specificity of this antibody (see Nakayama et al., (2001) Science, 292, pp 110-113, the disclosure of which is incorporated herein).

Using this antibody in chromatin immunoprecipitation (ChIP) experiments (Nakayama et al, (2000) Cell, 101, 307), it was discovered that the H3 Lys 9-methyl modification is specifically localized at the silenced chromosomal regions. H3 Lys 9 methylation and Swi6 were preferentially enriched at a marker gene (Kint2::ura4 1) inserted within the silenced mat2/3 chromosomal domain, compared with control ura4DS/E locus at the endogenous location. Similarly, H3 Lys 9 methylation was also preferentially enriched at the ura4 1 marker inserted within the highly repressed innermost repeat (imr1R::ura4 1) and the outer repeat (otr1R::ura4 1), but not at the weakly repressed central core (cnt1::ura4 1)of cen1. In addition, H3 Lys 9 methylation coincided with the presence of Swi6 at these regions (Partridge et al, (2000), Genes Dev., 14, 783). These findings suggest that H3 Lys 9-methyl modification and Swi6 are preferentially localized to silent chromosomal regions and that Swi6 localization is functionally dependent on H3 Lys 9 methylation.

Compared with the relatively high levels of Swi6 and H3 Lys 9 methylation at both Kint2::ura4 1 and otr1R::ura4 1 in wild-type cells, Swi6 and H3 Lys 9 methylation were absent in a clr4D strain at both loci. This result suggests that H3 Lys 9 is the physiological target of Clr4 HMTase activity and that Clr4 appears to be the exclusive in vivo H3 Lys 9-specific HMTase at mat and cen loci. In comparison with the in vitro result showing that only the SET domain is required for Clr4 HMTase activity, both the chromo-and SET domains are required for H3 Lys 9 methylation and Swi6 localization in vivo. Taken together, these results indicate that the chromodomain is presumably required for targeting Clr4 to the mat2/3 region and centromeres, whereas the SET domain and associated cysteine-rich regions of Clr4 constitute the catalytic site. The Swi6 levels at mat and cen in different clr4 mutant backgrounds were directly correlated with H3 Lys 9 methylation levels, further suggesting that Swi6 localization at silent chromosomal domains is functionally dependent on H3 Lys 9 methylation.

The importance of the in vivo analyses was further highlighted by observations that some mutations in Clr4 that decrease its HMTase activity in vitro do not substantially decrease H3 Lys 9 methylation and Swi6 localization in vivo. In addition, mutations in the SET domain and the NH 2-terminal cysteine-rich regions of Clr4 (G378S and R320H) greatly reduce H3 Lys 9 methylation and Swi6 localization at the mat locus; however, these mutations have moderate or negligible effects at cen1. These mutations also have weak effects on centromeric silencing compared with mating-type silencing. The results are consistent with the notion that enzymatic defects displayed by recombinant monomeric proteins in vitro can be “rescued” by functioning in the context of a multisubunit complex in vivo. Moreover, the functional organization of the mat2/3 region and centromeres may differ, and an additional factor(s) may help promote Clr4 activity at centromeres.

Mutations in the clr3 HDAC, which specifically deacetylates H3 Lys 14, affects silencing at mat and cen (S. I. S. Grewal, M. J. Bonaduce, A. J. S. Klar, Genetics 150, 563 (1998)). ChIP analysis demonstrated that a clr3-735 mutant partially defective in H3 Lys 14 HDAC activity displayed a moderate decrease in H3 Lys 9 methylation and Swi6 localization at otr1::ura4+, coincident with the apparent reduction in its HDAC activity. This result suggests that H3 Lys 14 acetylation inhibits Clr4 HMTase in vivo. To further investigate the functional interaction between Clr3 and Clr4, a double-mutant strain containing the clr3-735 and clr4R320H mutations was created, a clr4 mutation that had the least effect on H3 Lys 9 methylation at otr1R::ura4+. ChIP analysis of the double mutant demonstrated that H3 Lys 9 methylation and Swi6 localization were nearly abolished when compared with the single mutants. These findings indicate that Clr3 acts synergistically with Clr4 to effectively localize Swi6 to heterochromatic domains. In other words, deacetylation of H3 Lys 14 by Clr3 is required for H3 Lys 9 methylation by Clr4 and for Swi6 localization either indirectly, by altering Clr4 activity, or directly or both. These data also support the theory that residues neighboring Lys 9, and potentially their modification states, play an important role in establishment of the appropriate H3 Lys 9-methyl mark. Previous studies have shown that rik1+ affects silencing as well as Swi6 localization at silent loci. Computational analyses revealed that Rik1 contains b-propeller domains typically found within WD-40 repeat proteins and are theorized to participate in protein:protein interactions. A mutation in rik1 completely abolished H3 Lys 9 methylation and Swi6 localization at both mat and cen compared with wild type.

WD-40 proteins are involved in many aspects of chromatin remodeling and histone metabolism, such as chromatin assembly and acetylation or deacetylation of histones. Therefore, the b-propeller domains of Rik1 may form a complex with Clr4 to recruit its HMTase activity to heterochromatic regions and may play a role in coupling other transacting factors, such as Swi6 and histone deacetylases.

The possible role of Swi6 on Clr4-dependent methylation of H3 Lys 9 was also tested. Strains carrying swi6-115 (W269R) mutation that severely reduced Swi6 protein levels were used. As expected, Swi6 localization at both mat and cen was abolished as demonstrated by ChIP analysis. The swi6-115 mutation did not cause any detectable change in H3 Lys 9 methylation when compared with the wild-type strain. These data indicate that Swi6 is dispensable for Clr4 function and suggest that Swi6 acts down-stream of Clr4 H3 Lys 9 methylation. Collectively, the above results define a temporal sequence of events leading to establishment of the silenced chromatin state with regard to the covalent modifications of the H3 NH 2-terminal tail. HDACs and HMTases act cooperatively to establish a “histone code” that is then recognized by Swi6. More specifically, the HDACs (Clr6 and/or Hda1) deacetylate H3 Lys 9, whereas Clr3 deacetylates H3 Lys 14 before H3 Lys 9 methylation by the Clr4/Rik1 HMTase complex. Swi6 binding to the H3 Lys 9-methyl modification would then result in self-propagating heterochromatin assembly. Because the heterochromatin-binding domain of Swi6 was mapped to its chromodomain, it is most likely that this protein motif has evolved to recognize the H3 Lys 9-methyl modification.

It was recently shown that Swi6 remains associated with the mat2/3 region throughout the cell cycle where it acts as an important determinant of the epigenetic cellular memory, promoting inheritance of the silenced state. Because the mouse homolog of Swi6, M31, associates with Su(var)3-9, a similar inter-action between Clr4 and Swi6 is predicted. The close association of Clr4 enzymatic HMTase activity, followed by recruitment and binding of Swi6 to Lys 9 methyl “marks” in H3 through its chromodomain, suggests a pathway of epigenetic inheritance. The extent to which the chromo-domain of Clr4 recognizes H3 Lys 9-methyl marks is unknown, but it would provide the enzyme a means to bind chromatin as it performs subsequent methylation events. On the basis of the conservation of Clr4/SUV39H1 and Swi6/HP1 proteins and the presence of H3 Lys 9-methyl modification in higher eukaryotes, a similar mechanism may be responsible for higher order chromatin assembly in organisms ranging from fission yeast to humans. Considering the parallels between transcriptional repression by Polycomb group proteins in flies and mammals and silencing in fission yeast, it is likely that histone methylation coupled with histone deacetylation may help localize Polycomb in pathways that lead to the regulation of homeotic gene expression.

### EXAMPLE 3

Differential Sites of Histone H3 Methylation Marks the Active and Inactive Genes on the Human X Chromosome

Chromosomes in higher eukaryotes have historically been considered to consist of regions of euchromatin and heterochromatin, which are distinguished by the degree of condensation and level of transcriptional activity of the underlying DNA sequences. Certain regions of constitutive heterochromatin are found at or near specialized structures such as centromeres, and are comprised mostly of genetically inert repetitive sequences. In contrast, other regions that have the same primary DNA sequences can exhibit characteristics of either type of chromatin, suggesting that epigenetic factors, such as packaging of DNA by histones and chromatin-associated proteins, dictate the heterochromatin status at these loci.

One of the most dramatic examples of epigenetic silencing is the X chromosome inactivation seen in female cells of mammals. This process allows for dosage compensation of X-linked genes whereby one of the two copies of the X chromosome in female cells is randomly inactivated during embryonic development. Current evidence suggests that X inactivation is initiated by the up-regulation of the non-coding MIST transcript and its association in cis with the chromosome to be inactivated. Following its coating by XIST RNA, the inactive X chromosome acquires heterochromatic characteristics such as late replication timing, a condensed appearance (Barr body) in interphase cells, DNA methylation of CpG islands at house-keeping genes, and association with altered nucleosomes that are composed of hypoacetylated histones and enriched for the H2A variant MacroH2A. While the exact roles of these properties in the onset of X inactivation remain unclear, once the inactive state has been established, these epigenetic characteristics seem to act synergistically to maintain the remarkable stability of the inactive X through many cell divisions in the adult some

Recently, several publications showed that histone H3 methylation is important in the assembly of heterochromatin in mouse and S. pombe (Lachner et al., Nature 410, 116(2001); Bannister et al., Nature 410, 120 (2001) and Nakayama et al Science 292, 110 (2001)). The chromodomain of mouse HP1 (and Swi 6 in S. pombe) can bind to H3 methylated at lysine 9, and methylation of H3 at this site is thought to mark and recruit factors involved in heterochromatin assembly. In addition, acetylation and methylation of H3 at lysine 9 may be competing events in vivo (Cheung et al., Cell 103, 263 (2000)). Given that H3 is hypoacetylated on the inactive X chromosome, and that H3 lysine 9 methylation is important in heterochromatin assembly, the inactive X was investigated for enrichment for Lys9-methylated H3.

Using an antibody specific for Lys9-methylated H3, female human metaphase chromosomes were examined from a normal lymphoblast cell line (HH) by indirect immunofluorescence. In particular, a normal human female lymphoblast cell line (HH) and a female lymphoblast cell line which contains five X chromosomes (6061B) were grown, harvested and collected onto microscope slides with a Cytospin 3 centrifuge. Modified histones were detected by indirect immunofluorescence, essentially as described in detail elsewhere (Costanzi and J. R. Pehrson, Nature 393, 599 (1998)). Briefly, cells were incubated for one hour at 37° C. in a humid chamber with serial dilutions of the primary Lys9 methyl H3 or acetyl H4 antisera and washed in KCM (120 mM KCl, 20 mM NaCl 10 mM TRIS-CL, pH 8.0, 0.5 M EDTA, 0.1% Triton). The cells were then incubated for 30 min at room temperature with Cy3-conjugated, affinity-purified, donkey anti-rabbit IgG antibody (Jackson ImmunoResearch) diluted 1:40 in KCM. Cells were once again washed with KCM and fixed in 4% formaldehyde for 10 min at room temperature. Following a wash in sterile water chromosomes were counterstained with 4′,6-diamidino-2-phenylindole (DAPI), mounted in antifade (Vectashield) and viewed on a Zeiss Axiophot fluorescence microscope.

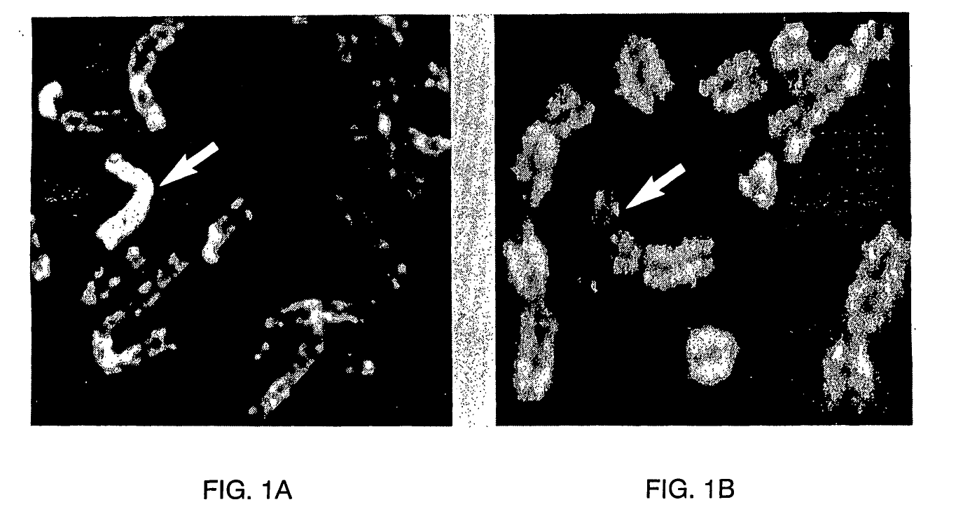

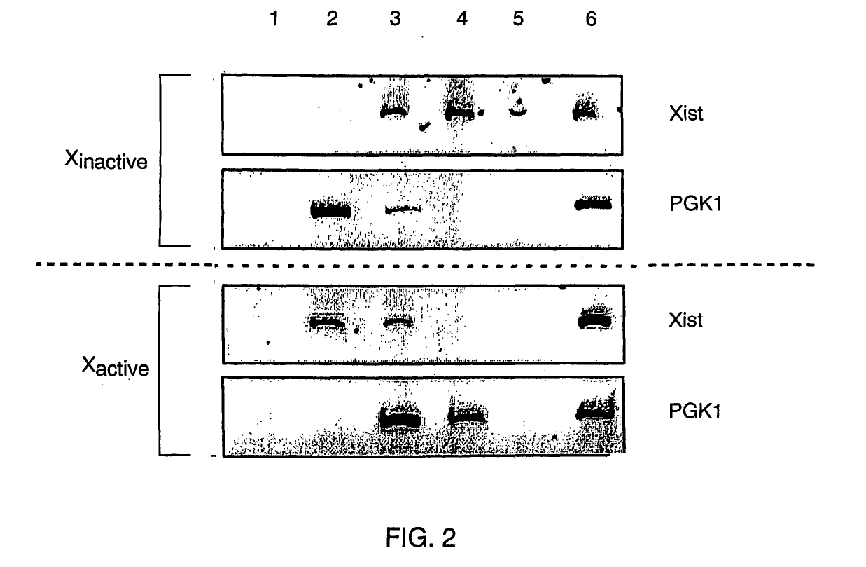

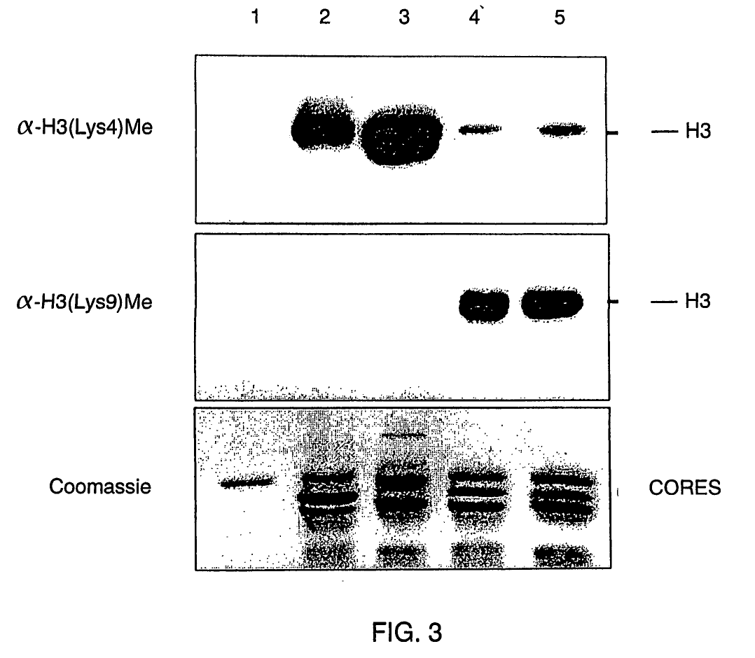

Indirect immunofluorescence using an antibody specific for Lys9-methylated H3 revealed that while most chromosomes have some regions of Lys9-methylated-H3, one chromosome in each metaphase spread is consistently more intensely and uniformly stained (FIG. 1A, white arrow). To test whether the chromosome enriched for Lys9-methylated H3 is the inactive X chromosome,. metaphase spreads from a cell line that contains five X chromosomes was also stained. In these cells, four out of the five X chromosomes are known to be inactivated and indeed four chromosomes of equal size show enriched staining by the Lys9-methyl H3 antibody. Together, these data suggest that while the H3 molecules on the inactive X are mostly hypoacetylated, they are highly methylated at lysine 9.

The N-terminus of histone H3 is post-translationally modified at multiple sites and lysine 4 of H3 is another documented site of methylation. However, in contrast to lysine 9, lysine 4 methylation has been correlated with active transcription. To further compare these two sites of H3 methylation on the inactive X chromosome, metaphase chromosomes were stained using an antibody specific for Lys4-methylated H3. Metaphase chromosomes from female cell lines HH and 6061B were incubated with Lys4 methyl H3 antiserum and the staining pattern was analyzed by indirect immunofluorescence as described earlier. Strikingly, this antibody intensely stains all chromosomes in the metaphase spread except for a single chromosome per spread (FIG. 1B). This unique chromosome is almost totally devoid of staining except for several ‘hot spots’ of H3 lysine 4 methylation. Staining of the metaphase spreads of the 5× cell line shows that four of the chromosomes are understained using the Lys4 methyl H3 antibody, suggesting that the hypo-H3 Lys4-methylated-chromosome(s) is the inactive X chromosome.

Closer examination of the inactive X chromosome shows that there are several distinct regions of this chromosome that exhibit intense staining with the Lys4 methyl H3 antibody. One region appears to be located at the pseudoautosomal region of the distal end of the p arm, another is located near Xq25-26 of the q arm, and fainter staining is occasionally seen around Xp11. With the exception of Xq25-26, the other regions of Lys4 methyl H3 staining on the inactive X correspond to the location of multiple genes known to escape inactivation (Carrel et al, Proc Natl Acad Sci USA 96, 14440 (1999)), and these data are therefore consistent with the idea that Lys4 methylation of H3 is associated with active gene expression.

The intense staining of the Xq25-26 region by the Lys4 methyl H3 antibody is puzzling since this region does not contain any known genes that escape X inactivation. However, it is intriguing that loss of heterozygosity at Xq25-26.1 is associated with advanced human ovarian carcinomas (see Choi,et al, Genes Chromosomes Cancer 20, 234 (1997)). Further studies of the enrichment of Lys4-methylated H3 at this region on the inactive X chromosome, and determining whether H3 Lys4/Lys9 methylation correlate with the expression level of the putative tumor suppressor gene(s) at this location will provide additional information on the link between histone H3 methylation and gene expression.

The understaining of the inactive X chromosome by the Lys4 methyl H3 antibody is similar, but not identical, to the staining pattern of this chromosome with antibodies against hyperacetylated form of H4. In this study, some staining at the telomeric region of the p arm of the inactive X chromosome was seen using the antibody against hyperacetylated H4, but not at the Xp11 and Xq25-26 regions. In contrast, previously published results showed that three regions on the inactive X chromosome (Xpter-22.2, Xp11.3-11.2, and Xq22) were stained with an antibody against hyperacetylated H4, but this was seen only in sodium butyrate-treated human cells. At present, the precise relationship between the regions stained by the hyperacetylated H4 and the Lys4 methyl H3 antibodies has not been clearly defined. Nevertheless, these data suggest that the inactive X chromosome is devoid of hyperacetylated core histones as well as Lys 4-methylated H3, but is enriched for H3 methylated at Lys9. Together, these combinations of histone modifications may be part of a series of concerted reactions that mediate transcriptional silencing of the inactive X.

To further establish the link between H3 Lys9 methylation and the inactive X chromosome, human female IMR9O interphase cells were examined by immunofluorescence in combination with Fluorescent In Situ Hybridization (FISH). In particular, IMR 90 (ATCC) cells were grown on coverslips for 24-48 hours then fixed in 4% formalehyde for 15 minutes at room temperature; the cells were then permeabilised in PBS containing 0.5% Triton-X for 4 min. on ice, washed in PBS and then washed in 2×SSC prior to RNA FISH. To preserve nuclear structures, cells were kept continously hydrated. RNA FISH hybridization and washes were performed essentially as described elsewhere (Lachner et al, Nature 410, 116 (2001)). Briefly, cells were hybridized with a XIST probe comprising a pool of four exon-derived DNA fragments spanning a total of 4.5 kb of sequence, labeled by nick translation with Spectrum Red or Green dUTP (Vysis, Downer Grove, Ill.). Following hybridization overnight at 37° C., standard washes for RNA FISH (ie 3× in 50% formamide/2×SSC and 3× in 2×SSC) were performed. The cells were then washed in PBS/0.5% BSA prior to performing immunofluorescence. Primary antibody was incubated for 1 hour at room temperature, cells were washed four time in PBS and the secondary antibody. (Texas red conjugated goat anti-rabbit antibody) was then applied for 1 hour at room temperature, followed by four washes in PBS. Nuclei were counterstained with DAPI. Images were acquired using a Zeiss Axioplan 2 fluorescence microscope with an Orca 2 CCD camera (Hamamatsu) and Improvision software (IPLab).

It is well established that the XIST transcript specifically localizes to the inactive X chromosome at interphase but not at metaphase, and localization of the Xist transcript can be detected by FISH analysis. The Lys9-methyl-H3 antibody preferentially stains a region that is heterochromatin-dense as indicated by co-localization with DAPI-dense regions. This condensed region also co-localizes with the XIST RNA signal, indicating that the chromosome enriched for Lys9-methylated H3 is indeed the inactive X. Consistent with the metaphase chromosome results, staining of human interphase cells with the Lys4 methyl H3 antibody shows that the region corresponding to the inactive X (based on DAPI dense staining and localization of the Xist RNA) is devoid of Lys4 methyl H3 staining. Intriguingly, a distinct dot enriched for Lys4 methyl H3 is seen within the region of negative staining for some of the cells. Whether this localized region of Lys4 methyl H3 staining corrresponds to the intensely stained regions seen in the metaphase chromosome is at present not known. Side by side comparisons of the Lys9 and Lys4 methyl H3 staining in interphase and metaphase cells further showed that the respective staining pattern of these two antibodies are almost reciprocal images. These results have led to the conclusion that lysine 4 and lysine 9 methylation of H3 are ‘reciprocal marks’ for transcriptionally active and inactive regions respectively, and hence the inactive X chromosome is hypomethylated at lysine 4 but is hypermethylated at lysine 9.

One resulting prediction is that the Lys4 and Lys9 methyl H3 antibodies would respectively enrich for active and inactive genes on the X chromosome by chromatin immunoprecipitation (ChIP). On the inactive X chromosome, the XIST gene is transcriptionally active whereas the PGK1 gene is silenced. Conversely, on the active X chromosome, the XIST gene is silent whereas the PGK1 gene is actively transcribed. To test the above hypothesis, two CHO somatic hybrid cell lines were used that contain either a single active or inactive human X chromosome in ChIP assays in order to examine the histone modifications associated with genes present on the active or inactive X chromosomes. Chromatin from these two cell lines were immunoprecipitated using antibodies against Lys9-methylated H3, Lys4-methylated H3, or Lys9/14-acetylated H3, and the immunoprecipitated DNA was PCR amplified using primers specific to the promoter regions of the human Xist and PGK1 genes.

In particular, Chromatin immunoprecipitation assays were done as described in Cheung et al, Mol Cell 5, 905 (2000). In this case, formaldehyde-fixed chromatin was harvested from CHO somatic cell hybrids containing either the active or inactive human X chromosome. Approximately 3×10⁶ cells-worth of sonicated chromatin were used per immunoprecipitation reaction with the antibodies indicated in the text. After extensive washing, reverse cross-linking, RNase A and proteinase K digestions, the immunoprecipitated DNA was analyzed by PCR using primers specific for the promoter regions of the human XIST and PGK1 genes (primer sequences and PCR conditions were derived from Gilbert and P. A. Sharp, Proc Natl Acad Sci USA 96, 13825 (1999)), and analyzed by polyacrylamide gel electrophoresis.

Consistent with previously published reports, the acetylated H3 antibody immunoprecipitated XIST DNA only from the inactive X chromosome, and the PGK1 DNA only from the active chromosome. Analogous to these results, the Lys4 methyl H3 antibody preferentially immunoprecipitated the XIST DNA from the cells actively expressing XIST (from the cells containing the inactive X chromosome) and the PGK1 DNA from the cells containing the active X chromosome. Therefore, both acetylated H3 and Lys4-methylated H3 are enriched at the actively transcribing loci on both the active and inactive X chromosomes. Immunoprecipitation using the Lys9 methyl H3 antibody showed reciprocal results to those obtained with the Lys4 methyl and acetyl H3 antibodies. In this case, XIST DNA was immunoprecipitated only from cells containing the active X chromosome whereas the PGK1 DNA was immunoprecipitated only from the inactive X chromosome.

Taken together, the chromatin immunoprecipitation data indicate that Lys4- or Lys9-methylated H3 are reciprocally associated with active and inactive genes irrespective of the chromosomal context (active versus inactive X). Moreover, combined with the immunofluorescence results, these data suggest that the two distinct methylation sites on H3 may mark regions of active and inactive chromatin, respectively.

The inactive X chromosomes is a well-studied paradigm for epigenetic regulation of gene expression and for linking specialized nucleosomal architecture with transcription silencing. As mentioned earlier, the entire inactive X chromosome seems to be largely devoid of hyperacetylated histones, and a core histone variant, MacroH2A, has been found to be enriched at the inactive X chromosome. Interestingly, none of the above modifications alone can account for the stability associated with this form of epigenetic regulation. Histone methylation, however, has recently been described as a more ‘stable’ epigenetic chromatin mark whose functions in X inactivation and as a potential cellular ‘memory’ marker have yet to be explored.

Very recently, methylation of histone H3 at lysine 9 has been defined to be an important modification for heterochromatin assembly, and as shown here, this modification is also enriched in the facultative heterochromatin of the inactive X chromosome. In contrast, methylation of-H3 at lysine 4 is complementarily absent in the inactive X chromosome, suggesting that methylation of H3 at these two distinct sites may be reciprocal, and that H3 molecules methylated at lysine 4 are preferentially associated with transcriptionally active genes whereas the opposite is true for H3 methylated at lysine 9. These findings provide strong evidence in support of the concept that specific modifications at specific sites on the histone amino-terminal tails can impart distinct characteristics, and perform different cellular functions.

While the precedence for H3 methylated at lysine 9 functioning to recruit chromatin-binding factors has been shown, it is still not clear how this modification may participate in the inactivation of the X chromosome. Analogously, H3 methylated at lysine 4 may function to recruit transcription-enhancing factors or to block the association of transcription-repressive factors; however, direct evidence for either of these possibilities is still lacking. The present ChIP assays only examined promoters of X-linked genes, but it is anticipated that H3. methylated at these two respective sites are genome-wide marks that demarcate chromosome domains. Active and inactive regions of the eukaryotic genome not only adopt contrasting chromatin structure (euchromatin versus heterochromatin), but also have been shown to occupy distinct intranuclear domains. Therefore, methylation of H3 at lysines 4 or 9 may dictate the spatial distribution of associated chromosome regions in transcriptionally permissive versus restrictive environment.

Equally intriguing is the question of which enzyme is responsible for the methylation reaction. While previous studies have shown that the hSuVar3-9 is a H3 lysine9-specific histone methyltransferase, fibroblasts derived from SuVar3-9 knock out embryos retain the enrichment of lysine 9 methylated at the inactive X chromosome.

### EXAMPLE 4

Parent-Specific Complementary Patterns of Histone H3 Lys9 And Lys4 Methylation at the Prader-Willi Imprinting Center

Imprinted genetic loci show differential expression of maternal compared to paternal alleles in some or all tissues at some or all stages of development. The functional differences between maternal and paternal alleles of imprinted genetic loci must reflect structural differences between maternal and paternal chromosomes in the regions containing these loci. The simplest models for establishment of these structural differences hold that chromosomal domains are marked differentially during oogenesis and spermatogenesis, and that these gametic imprinting marks are maintained after fertilization in somatic cells. However, there is no a priori reason to assume that the gametic imprinting marks are identical to the imprinting marks responsible for differential gene expression after fertilization. If the gametic marks are not the same as the somatic marks, there must be α-mechanism for reading the gametic marks and using their information to impose somatic imprinting marks.

The identity of the imprinting marks in mammals has been the subject of extensive speculation and experimental analysis. An appealing candidate imprinting mark is 5-methylcytosine in CpG dinucleotides. Many imprinted loci show parent-of-origin specific DNA methylation of imprinted regions, and some of these parent-specific DNA methylation marks are established during gametogenesis and maintained in somatic cells. A mechanism for replicating methylated CpG dinucleotides exists: maintenance DNA methyltransferases (DNMTs) recognize hemimethylated DNA (DNA methylated on only one strand) and add methyl groups to cytosine residues on the complementary strand. A frequently-cited experiment in support of cytosine methylation as the imprint is the observation that DNMT1 −/− embryos, which die early in embryonic development, show biallelic expression of some imprinted loci whose normal monoallelic expression is associated with cytosine methylation of the inactive allele.

Although abundant data are consistent with cytosine methylation as the imprint, several lines of evidence exist for additional or alternative molecular imprints. First, a number of imprinted genes, including mouse Mash2, human CDKN1C, and human UBE3A, show no regions of parent-specific DNA methylation. Second, some genes show evolutionary conservation of imprinting without showing conservation of differentially methylated regions, suggesting that the differential cytosine methylation may be a secondary consequence of a primary imprint. Third, Mash2 imprinting is not disturbed in the DNMT1 −/− mouse embryo. Finally, the promoter region of the imprinted SNRPN-gene in the Prader-Willi syndrome (PWS) imprinting center (IC) shows differential cytosine methylation in somatic tissues of mouse and human, and the region is heavily methylated in mouse oocytes but unmethylated in mouse sperm; however, El-Maarri et al. (Nature Genet. 27, 341 (2001)) have recently shown that this region is completely unmethylated in human oocytes, as in human sperm, so that the differential cytosine methylation must arise after fertilization.

These data lead to the conclusion that, at least for some imprinted genes and some imprinted regions, the structural difference between maternal and paternal alleles inherited from the gametes that leads to differential gene expression after fertilization must be something other than cytosine methylation. In principle, this structural difference might be a heritable covalent modification of DNA other than cytosine methylation, a DNA-associated protein that remains stably associated with either the maternal or the paternal chromosome from the gamete through somatic cell divisions, or a covalent modification of a DNA-associated protein that is inherited in a parent-specific fashion.

Histone modifications, especially acetylation, have previously been shown to mediate effects of a number of transcriptional regulatory proteins, presumably by changing chromatin structure to increase accessibility to other transcriptional factors. Unlike acetylated histones, which are quite labile, methyl groups attached to histones show a very low level of turnover, making histone methylation a good candidate modification in epigenetic processes such as imprinting. Accordingly, a histone modification that has recently been associated with the formation of stable silenced chromatin regions in Drosophila and fission yeast, (the methylation of histone H3 on Lys9) as been examined along with the methylation of histone H3 on Lys4, which has been correlated with transcriptional activity in Tetrahymena.

The Prader-Willi syndrome (PWS)/Angelman syndrome (AS) region in human chromosome 15q11-q13 contains at least 10 imprinted genes within a ˜2 Mb region (Nicholls et al, Trends Genet. 14, 194 (1998)); 8 of these genes are expressed exclusively from the paternal chromosome, and loss of the active paternal alleles of these genes causes PWS, characterized by infantile hypotonia, mild developmental delay, and later-onset hyperphagia and obesity. Loss of the active maternal allele of one gene in this region, UBE3A, causes AS, characterized by severe mental retardation, lack of speech, seizures, and easily provoked laughter. This region can exist in either of two mutually exclusive epigenetic states, the paternal state and the maternal state. Establishment of the paternal state requires a DNA segment, referred to as the PWS imprinting center (PWS-IC) that includes the SNRPN promoter in cis; establishment of the maternal state requires a DNA segment approximately 30 kb centromeric of the PWS-IC referred to as the AS-IC. The functions of the PWS-IC and AS-IC in establishing epigenetic states of this region are not known.

Chromatin prepared from stimulated lymphocytes of controls, PWS individuals (lacking a paternal copy of 15q11-q13 through deletion or imprinting defect), and AS individuals (lacking a maternal copy of 15q11-q13) was immunoprecipitated with antibodies specific for either H3 methylated on Lys9 or H3 methylated on Lys4. DNA recovered from the immunoprecipitation was assayed by PCR for sequences in the PWS-IC, including the SNRPN promoter, and for other sequences in the region. The maternal copy of the PWS imprinting center (present in PWS chromatin) was immunoprecipitated by anti-methyl Lys9 H3 antibody, while there was dramatically reduced precipitation of this sequence on the paternal copy (present in AS chromatin). This result correlates well with the observation that maintenance of silenced heterochromatin in both Drosophila and fission yeast requires the function of Lys9 histone H3 methyltransferases. The region of maternal-specific H3 Lys9 methylation extends approximately 0.6 kb 5′ and 0.5 kb 3′from the SNRPN promoter. Conversely, the paternal copy of the PWS-IC was immunoprecipitated by anti-methyl Lys4 H3 antibody. This sequence was not precipitated on the maternal copy. Previous reports of association of this modification with active chromatin are consistent with these findings.

Parent-specific differential association of methyl Lys9 H3 was not detected with the promoters of other imprinted genes in 15q11-q13, including ZNF127, NDN, MAGEL2, IPW, which are paternally-expressed, and UBE3A and ATP10C, which show tissue-specific maternal expression. Methyl Lys9 H3 was also not associated with the AS-IC. However, methyl Lys4 H3 was found to be specifically associated with the promoter region of the paternal allele of the paternally-active gene NDN. Parent-specific Lys4 methylation in lymphocyte chromatin was not detected for ZNF127, MAGEL2, IPW, UBE3A, or ATP10C.

Each of the modifications that has been described (cytosine methylation, histone H3 and H4 acetylation, histone H3 Lys4 methylation, and histone H3 Lys9 methylation) shows a distinct pattern of distribution and parent specificity. The PWS-IC, which overlaps the SNRPN promoter, shows the most extensive pattern of modification, with cytosine methylation and H3 Lys9 methylation on the maternal allele, and histone H3 and H4 acetylation as well as histone H3 Lys4 methylation on the paternal allele. The paternal SNRPN promoter region is also the site of a very prominent nuclease hypersensitive site that is not present on the maternal chromosome. The promoter region of NDN, which shows differential cytosine methylation, does not show either differential histone acetylation or differential H3 Lys9 methylation.

It is clear that the human PWS imprinting center lacks cytosine methylation in oocytes; therefore, this modification can not be the gametic imprint for the AS/PWS region. Among the parent-specific histone modifications of the PWS imprinting center, H3 and H4 acetylation, as well as H3 Lys4 methylation, also can not be the gametic imprint because sperm lack histones, so a paternal gametic imprint can not be a histone modification. Methyl Lys9 H3, however, is a potential candidate imprinting mark that could be imposed upon histones in the PWS imprinting center during gametogenesis. A maternal histone modification imprint would have the unique feature of undergoing programmed erasure during spermatogenesis, when histones are removed from chromatin and replaced by protamines.

1. A purified peptide comprising an antigenic fragment of the first 15 amino acids of the amino terminus of H3, said antigenic fragment comprising an amino acid sequence selected from the group consisting of:
ARTKQTAR (SEQ ID NO: 1), QTARKSTGV (SEQ ID NO: 2), QTARKSTGG (SEQ ID NO: 3), ARTKQTARGC (SEQ ID NO: 4), QTARKSTGVCG (SEQ ID NO: 5), and QTARKSTGGCG (SEQ ID NO: 6); and
amino acid sequences that differ from SEQ ID NO: 1-6 by one or more conservative amino acid substitutions.

2. The purified peptide of claim 1 wherein the peptide consists of ARTKQTARGC (SEQ ID NO: 4), QTARKSTGVCG (SEQ ID NO: 5) or QTARKSTGGCG (SEQ ID NO: 6).

3. An antibody that binds specifically to a peptide selected from the group consisting of ARTKQTARGC (SEQ ID NO: 4), QTARKSTGVCG (SEQ ID NO: 5) QTARKSTGGCG (SEQ ID NO: 6), AARKSAPVCG (SEQ ID NO: 16), SGGVKKPHKCG (SEQ ID NO: 17) and RHRKILRDCG (SEQ ID NO: 18).

4. The antibody of claim 3 wherein the peptide is selected from the group consisting of ARTKQTARGC (SEQ ID NO: 4), QTARKSTGVCG (SEQ ID NO: 5) and QTARKSTGGCG (SEQ ID NO: 6).

5. The antibody of claim 3, wherein the antibody specifically binds to the sequence ARTKQTARGC (SEQ ID NO: 4).

6. The antibody of claim 3, wherein the antibody specifically binds to the sequence QTARKSTGVCG (SEQ ID NO: 5).

7. The antibody of claim 3, wherein the antibody specifically binds to the sequence QTARKSTGGCG (SEQ ID NO: 6).

8. The antibody of claim 3, wherein the antibody is a monoclonal antibody.

9. The antibody of claim 3, wherein the antibody is labeled.

10. A fragment of the antibody of claim 4 that retains binding specificity for the antigenic fragment of claim 2.

11. A composition comprising the antibody of claim 4 and a diluent or pharmaceutically acceptable carrier.

12. A method of detecting transcriptionally active chromatin, said method comprising the steps of
contacting said chromatin with an antibody, wherein the antibody specifically binds only to methylated lysine 4 residues in H3 histones;
removing unbound and non-specific bond antibody from the sample; and
detecting the antibody bound to the sample.

13. The method of claim 12, wherein the detection step comprises contacting said antibody with a labeled secondary antibody wherein said secondary antibody is an anti-immunoglobulin antibody.

14. A method of detecting transcriptionally inactive chromatin, said method comprising the steps of
contacting said chromatin with an antibody, wherein the antibody specifically binds only to methylated lysine 9 residues in H3 histones;
removing unbound and non-specific bond antibody from the sample; and
detecting the antibody bound to the sample.

15. The method of claim 14, wherein the detection step comprises contacting said antibody with a labeled secondary antibody wherein said secondary antibody is an anti-immunoglobulin antibody.

16. A kit for detecting euchromatin and heterochromatin, said kit comprising an antibody that specifically binds to a peptide selected from the group consisting of ARTKQTARGC (SEQ ID NO: 4), QTARKSTGVCG (SEQ ID NO: 5), QTARKSTGGCG (SEQ ID NO: 6), AARKSAPVCG (SEQ ID NO: 16), SGGVKKPHKCG (SEQ ID NO: 17) and RHRKILRDCG (SEQ ID NO: 18).

17. The kit of claim 16 wherein the antibody specifically binds to a peptide selected from the group consisting of ARTKQTARGC (SEQ ID NO: 4), QTARKSTGVCG (SEQ ID NO: 5) and QTARKSTGGCG (SEQ ID NO: 6).

18. The kit of claim 16 comprising a first antibody that specifically binds to ARTKQTARGC (SEQ ID NO: 4), and a second antibody that specifically binds to QTARKSTGVCG (SEQ ID NO: 5) or QTARKSTGGCG (SEQ ID NO: 6),

19. A method of generating methyllysine 4 (histone)-specific antisera, said method comprising the steps of
chemically synthesizing a short polypeptide comprising the histone amino-acid sequence flanking the target methylated lysine;
conjugating the polypeptide to cationized bovine serum albumin; and
injecting the conjugated-peptide into rabbits or mice.

20. A method of detecting the presence of methylated H3 histones, said method comprises the steps of contacting histone proteins with an antibody, wherein the antibody specifically binds to H3 that is methylated at lysine 4 or lysine 9.

21. A method of detecting chromatin alterations that are associated with a disease state, said method comprising the steps of
isolating chromatin from both normal and diseased tissue to create a first and second pool of chromatin;
contacting the first and second pools of chromatin with an antibody selected from the group consisting of Methyl(K4)H3 and Methyl(K9)H3; and
comparing the staining pattern of the antibody bound chromatin isolated from normal tissue to the staining pattern of the antibody bound chromatin isolated from the diseased tissue.

22. The method of claim 21 wherein the chromatin comprises metaphase chromosomes.

23. A method of identifying nucleic acid sequences that are associated with a disease state, said method comprising the steps of
isolating chromatin from both normal and diseased tissue to create a first and second pool of chromatin;
immunoprecipitating the fragmented chromatin with an antibody selected from the group consisting of Methyl(K4)H3 and Methyl(K9)H3;
isolating DNA from the immunoprecipitated fragmented chromatin; and
comparing the DNA isolated from the first pool of chromatin to the DNA isolated from the second pool of chromatin.

24. The method of claim 23 further comprising the step of fragmenting the isolated chromatin before the immunoprecipitation step.

25. The method of claim 24 wherein the step of comparing the DNA comprises
immobilizing the DNA isolated from the first pool of chromatin onto a first solid surface;
immobilizing the DNA recovered from the second pool of chromatin onto a second solid surface;
probing the first and second solid surfaces with identical labeled nucleic acid sequences; and
identifying those sequences that bind only to the immobilized DNA isolated form the first pool of chromatin.


SEQUENCE LISTING (18 sequences)

SEQ ID NO: 1 - protein, 8 aa, Homo sapiens
  MOD_RES (4)..(4): METHYLATION
ARTKQTAR

SEQ ID NO: 2 - protein, 9 aa, Tetrahymena thermophila
  MOD_RES (5)..(5): METHYLATION
QTARKSTGV

SEQ ID NO: 3 - protein, 9 aa, Homo sapiens
  MOD_RES (5)..(5): METHYLATION
QTARKSTGG

SEQ ID NO: 4 - protein, 10 aa, Artificial
  synthetic peptide used to raise antibodies against the H3 amino terminus having MeLys at the fourth amino acid position.
ARTKQTARGC

SEQ ID NO: 5 - protein, 11 aa, Artificial
  synthetic peptide used to raise antibodies against the H3 amino terminus having MeLys at the fifth amino acid position.
QTARKSTGVCG

SEQ ID NO: 6 - protein, 11 aa, Artificial
  synthetic peptide used to raise antibodies against the H3 amino terminus having MeLys at the fifth amino acid position.
QTARKSTGGCG

SEQ ID NO: 7 - protein, 15 aa, Homo sapiens
ARTKQTARKSTGGKA

SEQ ID NO: 8 - protein, 15 aa, Homo sapiens
SGRGKGGKGLGKGGA

SEQ ID NO: 9 - protein, 13 aa, Tetrahymena thermophila
ARTKQTARKSTGV

SEQ ID NO: 10 - protein, 13 aa, Homo sapiens
  MOD_RES (4)..(4): METHYLATION
ARTKQTARKSTGG

SEQ ID NO: 11 - protein, 13 aa, Homo sapiens
  MOD_RES (9)..(9): METHYLATION
ARTKQTARKSTGG

SEQ ID NO: 12 - protein, 13 aa, Tetrahymena thermophila
  MOD_RES (4)..(4): METHYLATION
ARTKQTARKSTGV

SEQ ID NO: 13 - protein, 13 aa, Tetrahymena thermophila
  MOD_RES (9)..(9): METHYLATION
ARTKQTARKSTGV

SEQ ID NO: 14 - protein, 13 aa, Homo sapiens
  MOD_RES (5)..(5): METHYLATION
SGRGKGGKGLGKG

SEQ ID NO: 15 - protein, 13 aa, Homo sapiens
  MOD_RES (8)..(8): METHYLATION
SGRGKGGKGLGKG

SEQ ID NO: 16 - protein, 10 aa, Artificial
  synthetic peptide used to raise antibodies against the H3 amino terminus having MeLys at the fourth amino acid position
AARKSAPVCG

SEQ ID NO: 17 - protein, 11 aa, Artificial
  synthetic peptide used to raise antibodies against the H3 amino terminus having MeLys at the fifth amino acid position
SGGVKKPHKCG

SEQ ID NO: 18 - protein, 10 aa, Artificial
  synthetic peptide used to raise antibodies against the H3 amino terminus having MeLys at the fourth amino acid position
RHRKILRDCG